(19) United States
(12) Patent Application Publication
(10) Pub. No.: US 2010/0191038 A1
(43) Pub. Date: Jul. 29, 2010

(54) Devices and tools for treatment of urinary incontinence

(75) Inventors: Thomas P. Kubalak, Wayzata, MN (US); Christopher Deegan, North, St. Paul, MN (US); Daniel J. Dravis, Eau Claire, WI (US); Michele Watrin, Minneapolis, MN (US)

Correspondence Address: COLOPLAST A/S;Attention: Corporate Patents, Holtedam 1, DK-3050 Humlebaek, DK

(73) Assignee: COLOPLAST A/S, Humlebaek (DK)

(21) Appl. No.: 12/488,630

(22) Filed: Jun. 22, 2009

Related U.S. Application Data
(60) Provisional application No. 61/147,478, filed on Jan. 27, 2009.

(51) Int. Cl. A61F 2/02

(52) U.S. Cl. 600/30; 600/37

(57) ABSTRACT

Implantable medical devices provide support to a patient's urethra, surgical tools are utilized with the devices, and surgical methods are utilized to implant the devices in patients for treatment of urinary incontinence.

This application claims the benefit of U.S. Provisional Application Ser. No. 61/147,478 filed Jan. 27, 2009.

### FIELD OF THE INVENTION

This document relates generally to medical devices. More particularly, this document relates to implantable medical devices, surgical tools for use with the devices, and surgical methods of implanting the devices in a patient, for treatment of urinary incontinence.

### BACKGROUND

Suburethral sling devices to treat urinary incontinence have been proposed in recent years, but such known devices may have several disadvantages. For example, tools and methods for implanting suburethral sling devices often lead to difficulties in securing them into position with optimal degrees of tension. Indeed, the implantation of suburethral sling devices for the treatment of incontinence may be complex and time consuming, and may produce suboptimal clinical outcomes.

### SUMMARY

This document describes medical devices, surgical tools, and surgical methods that may address and alleviate the aforementioned deficiencies in the medical art.

This document also describes medical devices, surgical tools, and surgical methods that may be effective and substantially easier to use than known devices, tools, and methods for treating incontinence.

Accordingly, this document describes novel implantable medical devices that provide urethral support, novel surgical tools that are utilized with the devices, and novel surgical methods to implant the devices in patients.

In one aspect, an implantable device comprises a body member, a plurality of elongated extension members—each extending from the body member and having an end portion—and a plurality of sutures, each being connected to, respectively, the end portion of each of the plurality of elongated extension members. In another aspect, the implantable device further comprises a plurality of sleeves, each removably covering at least a portion of the plurality of elongated extension members, respectively. In another aspect, the implantable device further comprises a plurality of tips, each removably covering at least a portion of the plurality of sleeves and at least an end portion of the plurality of elongated extension members, respectively.

In another aspect, an implantable device comprises a body member having a top portion, a bottom portion, and a longitudinal axis extending through the top and bottom portions. The device also comprises at least first and second elongated extension members on opposite sides of the body member—each having an end portion, extending from the top portion of the body member, and having a longitudinal axis extending therethrough. The device also comprises at least third and fourth elongated extension members—each having an end portion, extending from the bottom portion of the body member, and having a longitudinal axis extending therethrough. The device also comprises at least one suture, connected to at least one of the elongated extension members, respectively, at the end portion thereof. The device also comprises at least one sleeve, covering a portion of at least one of the elongated extension members with the suture extending outwardly therefrom, respectively. The device also comprises at least one tip, covering and connected to the at least one sleeve and the elongated extension members, respectively, at the end portion thereof, with the suture extending outwardly therefrom, respectively. In another aspect, the body member has a generally trapezoidal shape. In another aspect, the longitudinal axis of the at least first elongated extension member is approximately colinear with the longitudinal axis of the at least second elongated extension member. In another aspect, the longitudinal axis of the at least third elongated extension member is approximately parallel to the longitudinal axis of the at least fourth elongated extension member. In another aspect, at least one sleeve is color-coded. In another aspect, at least one suture forms a loop.

In another aspect, a surgical tool comprises a shaft having a proximal end, an intermediate hook-shaped portion, a distal end opposite the proximal end, and at least one T-shaped slotted portion in the distal end of the shaft. In another aspect, the surgical tool further comprises at least one L-shaped slotted portion in the distal end of the shaft.

In another aspect, a surgical method, for use with an implantable device having a body member including a top portion and a bottom portion, and at least first, second, third, and fourth elongated extension members, each having an end portion, comprises placing the at least first, second, third, and fourth elongated extension members in a patient so that the body member is proximate to the patient's urethra. The at least first and second elongated extension members are secured through opposite ones, respectively, of the patient's obturator foramen, so that the at least first and second elongated extension members provide desired elevation of the body member connected thereto, thereby elevating the patient's urethra. The at least third and fourth elongated extension members are secured through incisions above a pubic symphysis on a right side and a left side of the patient, respectively, so that the at least third and fourth elongated extension members provide desired tension on the body member of the implantable device connected thereto, thereby compressing the patient's urethra. In another aspect, the implantable device of the surgical method further comprises at least first, second, third, and fourth sutures, connected to the end portion of the at least first, second, third, and fourth elongated extension members, respectively. In another aspect, the implantable device of the surgical method further comprises at least first, second, third, and fourth sleeves, removably covering at least the end portion of the at least first, second, third, and fourth elongated extension members, with the sutures extending outwardly therefrom, respectively. In another aspect, the implantable device of the surgical method further comprises at least first, second, third, and fourth tips, connected to the sleeves over the end portion of the elongated extension members, with the sutures extending outwardly therefrom, respectively. In another aspect of the surgical method the steps of placing the elongated extension members comprise use of a surgical tool having at least one T-shaped slotted portion therein. In another aspect of the surgical method, the at least first and second elongated extension members are tensioned to elevate the body member of the implantable device, thereby elevating the patient's urethra. In another aspect of the surgical method, the at least third and fourth elongated extension members are tensioned to tension the body member of the implantable device, thereby compressing the patient's urethra.

In another aspect, a surgical method, for use with an implantable device having a body member and at least first, second, third, and fourth elongated extension members, comprises placing the body member at a location in a patient that is proximate to, and approximately centered on, the patient's urethra. The at least first and second elongated extension members are secured through opposite ones, respectively, of the patient's obturator foramen, so that the at least first and second elongated extension members inhibit lateral shifting of the body member away from the location in the patient that is proximate to and approximately centered on the patient's urethra and also provide desired elevation of the body member connected thereto, thereby elevating the patient's urethra. The at least third and fourth elongated extension members are secured through incisions above a pubic symphysis on a right side and a left side of the patient, respectively, so that the at least third and fourth elongated extension members provide desired tension on the body member connected thereto, thereby compressing the patient's urethra.

### BRIEF DESCRIPTION OF THE DRAWINGS

FIG. 1 is an illustration of one embodiment of an implantable device.

FIG. 1A is a magnified illustration of a portion of the implantable device shown in FIG. 1.

FIG. 2 is an illustration of one embodiment of a surgical tool.

FIG. 2A is a magnified illustration of a portion of the tool shown in FIG. 2.

FIG. 3 is a flowchart of one embodiment of a surgical method.

### DETAILED DESCRIPTION

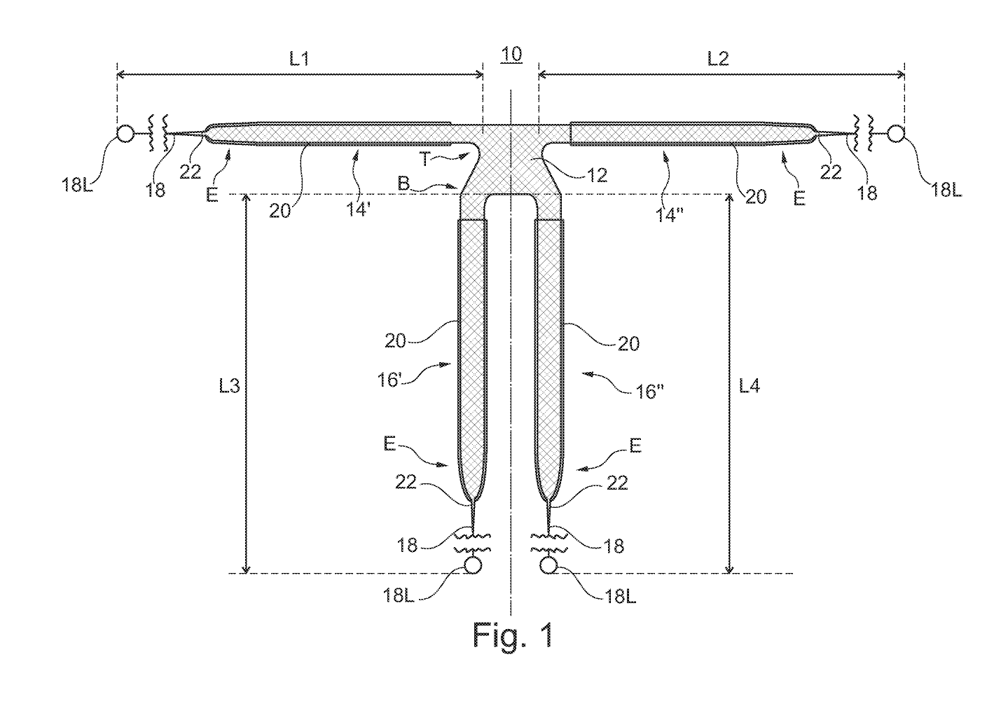

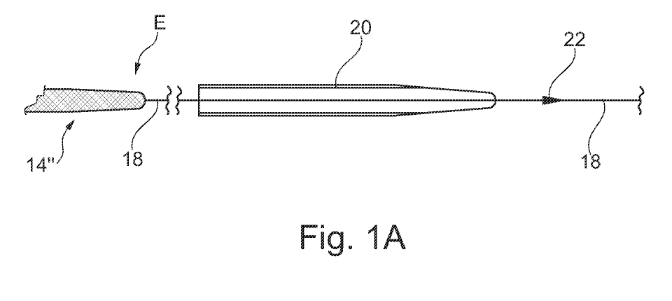

One embodiment of an implantable device 10 (or, “device 10”) is illustrated in FIG. 1. Therein, device 10 includes a body member 12, a first elongated extension member or arm 14′, a second elongated extension member or arm 14″, a third elongated extension member or arm 16′, and a fourth elongation extension member or arm 16″. Arms 14′-16″ have, respectively, longitudinal axes L₁-L₄ that are shown offset from their centers for clarity in the drawing. Arms 14′-16″ are oriented to provide both anchoring of device 10 in a patient's anatomy and support of the patient's urethra as will be further described.

In FIG. 1, body member 12 has a generally trapezoidal shape, with arms 14′ and 14″ extending laterally away from, and generally parallel to, a top portion T of body member 12, and with legs 16′ and 16″ extending longitudinally away from, and generally transverse to, a bottom portion B of body member 12. It is to be understood, however, that arms 16′ and 16″ could be in any orientation with respect to each other, whether parallel, substantially so, or not parallel at all. Ultimately, orientation of any of the elongated extension members could be chosen with respect to suitability for each patient's unique anatomy.

As shown in FIG. 1, approximate dimensions of device 10 are as follows: in body member 12, lengths of top portion T and bottom portion B are about 6.1 cm and 7.0 cm, respectively; lengths of arms 14′ and 14″ are about 19.5 cm; and lengths of arms 16′ and 16″ are about 25.0 cm. An overall thickness of mesh material (as described below) comprising body member 12 and arms 14′-16″ is about 0.6 mm. It is to be appreciated, however, that such dimensions of these components of device 10 may vary depending on a particular type of material used for its manufacture and also on a particular anatomy of a patient into whom it is being implanted. Thus it is to be understood that a narrowest end (or, top portion T) of body member 12, from where arms 14′ and 14″ oppositely extend, should be capable of fitting between the patient's opposing pubic rami and obturator foramen. In this regard, it will be appreciated that if a width of the narrowest end of body member 12 exceeds a width between the patient's pubic rami, then there may be a possibility that material of device 10 could fold, bulge, or otherwise deform and thus compromise a successful treatment outcome. However, a widest end (or, bottom portion B) of body member 12 serves to support the patient's urethra when implanted; thus, a larger surface of the widest end—relative to the narrowest end of body member 12—could provide an approximately even distribution of pressure and provide a desirable compression of the patient's urethra as will be further described.

Sterilizable materials that are suitable for use in constructing body member 12 and arms 14′-16″ of device 10 could include synthetic materials such as meshes and the like, natural tissues such as tissues harvested from an animal, a cadaverous source, or the patient himself or herself, or any suitable combinations of synthetic and natural materials. For example, body member 12 and arms 14′-16″ could be fabricated from a knitted, monofilament polypropylene mesh material having desired wales per inch, courses per inch, thickness, density, pore sizes, strength, flexibility, and stiffness. Such a material could be, for example, an “ARIS” brand mesh material that is commercially available from Coloplast A/S.

With reference to FIG. 1A and continued reference to FIG. 1, the embodiment of device 10 therein illustrated includes first, second, third, and fourth sutures 18, that are each connected to end portions E of arms 14′-16″, respectively. The connections are made by tying the suture to end E of the mesh material at approximately 0.5 cm or about 3 pores from an endmost portion thereof; and sutures 18 are provided by, for example, “POLYDEK”® brand surgical suture material that is commercially available from Teleflex Medical. In this example embodiment, sutures 18 each have a total end-to-end length of approximately 17 cm and are each formed into a loop 18L. Also, first, second, third, and fourth sleeves 20 removably cover end portions E of arms 14′-16″, respectively. During surgery, sleeves 20 may advantageously reduce friction between device 10 and the patient's tissues as device 10 is being implanted. In this embodiment, connections of sleeves 20 to arms 14′-16″ are made via tips 22 as described below. Sleeves 20 may be manufactured from any suitable, sterilizable polyethylene material. Optionally, any sleeve 20 could be color-coded. For example, sleeves 20 corresponding to arms 14′ and 14″ could be clear, while sleeves 20 corresponding to arms 16′ and 16″ could have a translucent blue color to aid in visual identification during surgery. Further, and as briefly aforementioned, this embodiment of device 10 includes first, second, third, and fourth tips 22, that cover at least a portion of sleeves 20 corresponding to, and in turn covering, end portions E of arms 14′-16″, respectively—with sutures 18 extending outwardly therefrom. A presence of tips 22 assists the elongated extension members or arms of device 10 in following a path through the patient's tissue made by a surgical tool during surgical implantation of device 10 as described below. Also, in this embodiment, tips 22 are relatively pointed or “necked-down”—thereby tightly surrounding ends E of arms 14′-16″ of device 10 and reinforcing connections of sutures 18 thereto. In this example embodiment, tips 22 are securely joined to sleeves 20 at ends E of device 10 by way of a so-called “heat staking” or “heat sealing” process. In this regard, with continued reference to FIG. 1A, conical, hollow tip 22 is placed over sleeve 20, suture 18, and elongated extension member 14″. Tip 22 is then compressed by use of pressure from a heated die (not illustrated). A combination of heat and pressure over time in the heated die effectively melts at least portions of tip 22 and sleeve 20, but not member 14″ or suture 18. During such time under heat and pressure, in a heating cycle, the melted portions of tip 22 and sleeve 20 become semi-liquid and flow through pores in member 14″. Thereafter, in a cooling cycle, those melted portions of tip 22 and sleeve 20 cool and stop flowing, thereby securely joining these components as shown in the example of FIG. 1. This process is performed with respect to all members 14′-16″, in this example of manufacturing device 10. It is to be appreciated that this process provides, advantageously, relatively flatter cross-sections or reduced profiles in the tips as compared to their original, relatively projectile-like shapes before being so processed. Specifically, the resulting reduced profiles may be beneficial in minimizing tissue disruption in the patient during implantation surgery when ends E of arms 14′-16″ are being passed through the patient's body as described below. Tips 22 may be fabricated from, for example, any suitable, sterilizable polymer material.

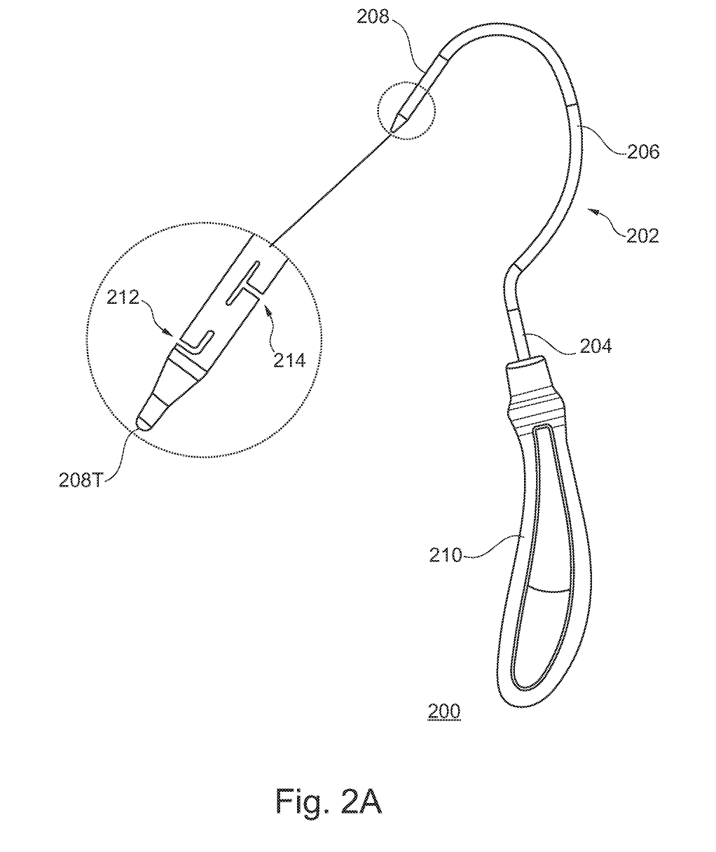

Turning, now, to FIGS. 2 and 2a, one embodiment of a surgical tool 200 (or, “tool 200”) is illustrated for surgical placement of device 10 as will be further described. In this example embodiment, tool 200 includes a shaft 202 having a proximal end 204, an intermediate hook-shaped portion 206, and a distal end 208 opposite proximal end 204. In this embodiment, shaft 202 has a generally cylindrical cross-section; but it could be formed into any desired geometry (e.g., oval, square, hexagonal, etc.). A handle 210 is securely connected to proximal end 204 of shaft 202. Handle 210 is, preferably, ergonomically shaped for comfort in a hand of, and for reliability and ease of use by, a physician. But like shaft 202, handle 210 could be formed into any suitable or desired shape. In this embodiment of tool 200, distal end 208 includes recessed apertures or slotted portions for removably securing or connecting device 10 to tool 200 in a surgical method as described below. Specifically, and as shown in FIG. 2A, in this embodiment distal end 208 includes both an L-shaped slot 212 and a T-shaped slot 214 for receiving and removably securing therein or connecting thereto suture loop 18L of device 10. With additional reference to FIG. 1, it is to be understood that slots 212 and 214 are configured to readily and easily capture or “grab” suture loops 18L. Thereby, slots 212 and 214 facilitate pulling, pushing, and placement of corresponding end portions E of elongated extension members 14′-16″ to which they are attached—via loops 18L—at various times during surgery to implant device 10. The elongated extension members could then be disconnected or detached from tool 200 by removing loop 18L from slot 212. It is to be understood that a particular tool 200 could include either slot 212 or slot 214, or a combination of them, in any orientation along distal end 208 of shaft 202 depending upon a physician's preference or a particular intended use of tool 200. Further, and as described below, it is to be appreciated that provision and use of an L-shaped slot may be preferred for an “inside-out” passage of end 208 of tool 200, while a T-shaped slot may be preferred for either an “inside-out” or “outside-in” passage of end 208 during surgical implantation of device 10. In this example, both L-shaped slot 212 and T-shaped slot 214 comprise connected first and second passages or channels in shaft 202, each having a width of about 0.38 mm. L-shaped slot 212 has a first channel length (i.e., from an outside surface of shaft 202, inwardly) of about 2.0 mm, and a second channel length (i.e., along shaft 202) of about 2.5 mm. T-shaped slot 214, like slot 212, has a first channel length (i.e., from an outside surface of shaft 202, inwardly) of about 2.0 mm, but a second channel length (i.e., along shaft 202) of about 4.7 mm. In the example embodiment shown in FIGS. 2-2A, tool 200 also includes a generally conical, pointed tip 208T at an endmost portion of distal end 208, to facilitate piercing and passage of distal end 208 through an obturator foramen of a patient and further through the patient's skin, to protrude outwardly from a skin exit location on the patient thereby created in a surgical method as described below. Shaft 202 of surgical tool 200 could be fabricated from 17-4 stainless steel; and handle 210 could be fabricated from any suitable, sterilizable materials such as, for example, santoprene and polycarbonate. It is to be understood that tool 200 could ultimately have any advantageous shapes, configurations, and dimensions to carry out an implantation surgery on a particular patient by a particular physician as will be described below. Thus, for example, a curvature of intermediate hook-shaped portion 206—or, even of an entire length of shaft 202—could be provided in many different forms, also with varying radii of curvature, as may be desired for a particular tool 200 and so that a physician could perform implantation surgery in an acceptable manner through desired tissue pathways in a patient as hereinafter described.

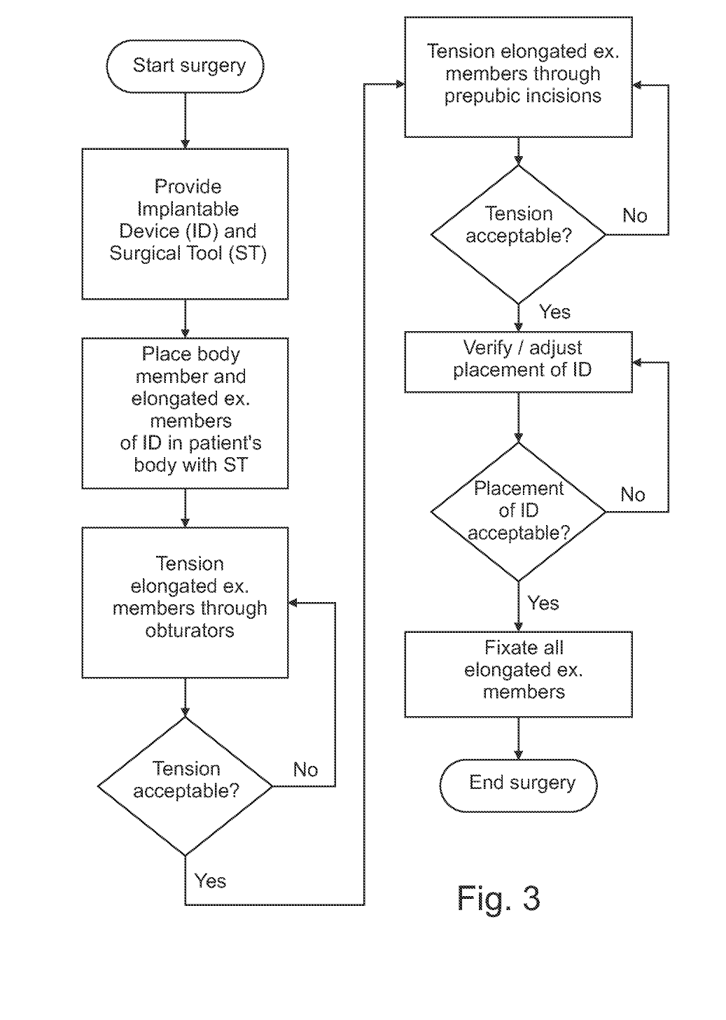

With reference, now, to FIG. 3, one embodiment of a method for treatment of urinary incontinence (or, “UI”) will now be described that utilizes the aforedescribed examples of an implantable device and a surgical tool. It is to be understood that FIG. 3 is a summary flowchart of one embodiment of this method.

In this embodiment of a method for treatment of UI, implantable device 10 and surgical tool 200 are provided to a physician. A vertical perineal incision is made in a midline of a patient, dissecting to isolate the patient's ventral urethra and pubic rami bilaterally, and leaving a bulbospongiosus muscle of the patient intact but exposing the bulbospongiosus muscle and taking it partially off the patient's perineal body. Suture loop 18L associated with one of at least first and second elongated extension members (e.g., arm 14′) is removably attached to tool 200 via L-shaped slot 212 or T-shaped slot 214. For an obese patient, L-shaped slot 212 could later provide easier detachment of loop 18L, as described below, than T-shaped slot 214. By gripping handle 210 of tool 200 and pushing tool 200 forward, tip 208T is then passed by the physician through the perineal incision from medial to lateral and through an upper aspect of an obturator foramen on the patient's right side as an inside-out movement. Tip 208T is then further passed and advanced so that it exits through and protrudes outwardly from a skin exit location on the patient's right side in a vicinity of that obturator foramen. Optionally, the physician could determine a location of tip 208T, by, e.g., externally palpating its presence under the patient's skin; and then the physician could make a corresponding skin incision to facilitate emergence of tip 208T at the skin exit location. Suture loop 18L is then detached from slot 212 or 214, thereby detaching end E of arm 14′ from tool 200; and tool 200 is removed from the patient. A portion of end E of arm 14′ is then pulled outwardly from the skin exit location on the patient's right side, to later facilitate tensioning of device 10 as will be described. This process is repeated on the patient's contralateral side (in this example, the patient's left side). Accordingly, suture loop 18L associated with another of the at least first and second elongated extension members (e.g., arm 14″) is removably attached to tool 200 via L-shaped slot 212 or T-shaped slot 214, again with L-shaped slot 212 providing easier detachment of loop 18L for an obese patient. By gripping handle 210 and pushing tool 200 forward, tip 208T is then again passed by the physician through the perineal incision from medial to lateral and through an upper aspect of an obturator foramen—this time, for example, on the patient's left side—as an inside-out movement. Tip 208T is then further passed and advanced so that it exits through and protrudes outwardly from a skin exit location on the patient's left side in a vicinity of that obturator foramen. Optionally, as aforementioned, the physician could determine a location of tip 208T by, e.g., palpation; and then the physician could make a corresponding skin incision to facilitate emergence of tip 208T from the skin. Loop 18L is then detached from slot 212 or 214, thereby detaching end E of arm 14″ from tool 200; and tool 200 is again removed from the patient. As with arm 14′, a portion of end E of arm 14″ is then pulled outwardly from the skin exit location on the patient's left side to facilitate later tensioning. An incision above the patient's pubic symphysis, and lateral to the midline, on both the right and left sides of the patient is then made. By gripping handle 210 of tool 200 and pushing tool 200 forward, tip 208T is then passed prepubically, through the incision above the pubic symphysis on, e.g., the right side of the patient, and further out through the perineal incision lateral to the patient's urethra. Suture loop 18L of, e.g., arm 16′ in device 10 is then attached to T-shaped slot 214 of tool 200. Tool 200 is then pulled backwardly, with tip 208T of tool 200 thus passing backwardly through a path from the perineal incision to the incision above the pubic symphysis on the right side of the patient and outwardly therefrom, so that tip 208T of tool 200 exits through that incision above the pubic symphysis. Loop 18L is then detached from slot 214, thereby detaching end E of arm 16′ from tool 200. Tool 200 is then removed from the patient. A portion of end E of arm 16′ is then pulled outwardly from the incision above the pubic symphysis on the patient's right side, to later facilitate tensioning of device 10 as will be described. This process is repeated on the patient's contralateral side (in this example, again, the patient's left side). By again gripping handle 210 of tool 200 and pushing tool 200 forward, tip 208T is passed by the physician prepubically, through the incision above the pubic symphysis on, e.g., the left side of the patient, and further out through the perineal incision lateral to the patient's urethra. Suture loop 18L of, e.g., arm 16″ in device 10 is then attached to T-shaped slot 214 of tool 200. Tool 200 is then pulled backwardly, with tip 208T of tool 200 thus passing backwardly through a path from the perineal incision to the incision above the pubic symphysis on the left side of the patient and outwardly therefrom, so that tip 208T of tool 200 exits through that incision above the pubic symphysis. Loop 18L is then detached from slot 214, thereby detaching end E of arm 16″ from tool 200; and tool 200 is again removed from the patient. As with arm 16′, a portion of end E of arm 16″ is then pulled outwardly from the incision above the pubic symphysis on the patient's left side, to facilitate tensioning. Tensioning of first and second elongated extension members (e.g., arms 14′ and 14″) is then optionally performed for a particular patient by simultaneously pulling on their ends E outside the patient's body to elevate body member 12 of device 10 connected thereto. Thereby, the patient's urethra is elevated as may be desired for that patient. Similarly, tensioning of third and fourth elongated extension members (e.g., arms 16′ and 16″) is then optionally performed by simultaneously pulling on their ends E outside the patient's body to elevate body member 12 of device 10 connected thereto. Thereby, the patient's urethra is compressed as may be desired. Body member 12 is optionally inspected to verify its placement at the patient's urethra, and a cystoscopy is optionally performed to ensure that proper coaptation of the patient's urethra has been achieved. According to results of the cystoscopy, further tensioning of the first, second, third, and fourth elongated extension members is optionally performed by again pulling on their ends E outside of the patient. The first, second, third, and fourth sleeves, tips, and sutures—corresponding to the first, second, third, and fourth elongated extension members—are then removed and discarded by any suitable technique such as by cutting ends E at a point between body member 12 and a portion of tips 22 that is most proximal to body member 12, thereby leaving inside and outside the patient only body member 12 of device 10 and the elongated extension members extending therefrom. Remaining ends E of elongated extension members 14′-16″ are then, for example, individually trimmed at and fixated in respective locations of subcutaneous layers in the patient. The fixation of ends E in the patient could be accomplished by any suitable means such as via suturing. Finally, the perineal incision, and the incisions above the pubic symphysis on the patient's right and left sides, are closed using any suitable surgical technique such as, for example, by use of sutures or “Dermabond” closure material. It is to be appreciated that following surgery, the mesh material of device 10 typically engages surrounding tissue in the patient with fibroblast infiltration possibly providing additional fixation of arms 14′-16″ and body member 12 in the patient.

It is to be particularly appreciated and understood that fundamentally, treatment of UI described herein utilizes a suitable implantable device to provide both desired elevation and compression of a patient's urethra. This desired effect on the urethra may be basically achieved, regardless of a particular device, tool, or method, by providing an implantable device having a body member defining a top portion and a bottom portion, at least first and second elongated extension members, and at least third and fourth elongated extension members. The first and second elongated extension members are placed in the patient and secured by way of passage through, respectively, the patient's opposing obturator foramen to provide substantial fixation of the body member of the device in spatial relationship to the patient's urethra. Separately and distinctly, the third and fourth elongated extension members are placed in the patient prepubically, above the pubic symphysis, to provide desired compression of the urethra.

It is also to be particularly appreciated and understood that one embodiment of treatment of UI described herein advantageously makes use of a novel method whereby the physician actually begins the implantation surgery by placing the device at a location in the patient where it is desired to finally reside in the patient. Specifically, the method places the body member at a location that is proximate to, and approximately centered on, the patient's urethra. The elongated extension members that are secured through opposite obturator foramen act to inhibit lateral shifting of the body member away from the location that is proximate to and centered on the patient's urethra, while also providing desired elevation of the body member connected thereto, thereby elevating the patient's urethra. The elongated extension members that are secured through incisions above a pubic symphysis on a right side and a left side of the patient, respectively, provide desired tension on the body member connected thereto, thereby compressing the patient's urethra.

It is also to be understood that the devices, tools, and methods described herein may satisfy a need in the art for treatment of UI in which the devices may be easier to surgically secure into position (or, as used here throughout, “fixate”) and that may further provide an optimal degree of urethral support to thus effectively treat UI, as compared to known devices, tools, and methods. The devices, tools, and methods described herein may additionally satisfy a need in the art for treatment of UI in which the devices may be simply constructed utilizing known implant materials—whether they are synthetic materials, natural tissues, or combinations thereof, and in which the devices and tools may be easily manipulated in surgery. The devices, tools, and methods described herein may satisfy yet a further need in the art for treatment of UI in which the devices define a suburethral portion operatively positioned to support a patient's urethra, with the devices remaining in position via use of (i) at least a pair of elongated extension members (or “arms”) that are secured through patients′ obturator foramen and (ii) at least another pair of elongated extension members (or “arms”) that are secured subcutaneously above patients' pubic symphyses. The devices, tools, and methods described herein may also satisfy a need for treatment of urinary incontinence that provides opportunities to efficiently adjust tensioning of the implanted devices.

Although the invention has been described in terms of particular embodiments and applications, one of ordinary skill in the art, in light of the teaching, can generate embodiments and modifications without departing from the spirit of, or exceeding the scope of, the claimed invention. For example, it will be clear to one of ordinary skill in the art how to apply the inventive concepts disclosed herein to the treatment of both male and female incontinence. Accordingly, it is to be understood that the drawings and descriptions herein are proffered by way of example to facilitate comprehension of the invention and should not be construed to limit the scope thereof. For instance, any number of elongated extension members, beyond the two pairs of arms illustrated and described herein, may be provided to attain the novel aspects of the present invention. Specifically, and as aforementioned, the present invention provides a novel combination of placement of extension members through obturator foramen (for securing the implantable device relative to the urethra) and above the pubic symphysis, prepubically, for compression of the urethra. Thus, for example, a total of eight arms could be provided in a particular embodiment of the present invention—e.g., two pairs of arms (4 arms) for placement through the obturator foramen and two pairs of arms (4 arms) for placement prepubically, above the pubic symphysis. Likewise, the body member of the implantable device could be of any desired shape or configuration to achieve these objectives and thereby effectively treat the patient's incontinence.

Furthermore, additional surgical steps could be performed (e.g., initially placing the patient in a slightly exaggerated lithotomy position and inserting a 16 Fr foley catheter, etc.) depending upon preferences of a particular physician and standard surgical protocols of a particular institution.

While the present invention has been particularly shown and described with reference to the accompanying specification and drawings, it will be understood however that other modifications thereto are of course possible; and all of which are intended to be within the true spirit and scope of the present invention. For example, surgical tools in forms of trocars and cannulas could be equally utilized, instead of the substantially solid, needle-like surgical tool shown and described herein. It should be appreciated that (i) components, dimensions, and other particulars of aforedescribed example embodiments may be substituted for others that are suitable for achieving desired results, (ii) various additions or deletions may be made thereto, (iii) features-of the foregoing examples may also be made in various combinations thereof, and (iv) certain method steps may be performed in various sequences. It is also to be understood in general that any suitable alternatives may be employed to provide devices, tools, and methods for treatment of urinary incontinence.

Lastly, choices of compositions, sizes, and strengths of various aforementioned elements, components, and steps all depend upon intended uses thereof. Accordingly, these and other various changes or modifications in form and detail may also be made, again without departing from the true spirit and scope of the invention as defined by the appended claims.

What is claimed is:

1. An implantable device, comprising:
a body member;
a plurality of elongated extension members, each (i) extending from said body member and (ii) having an end portion; and
a plurality of sutures, each being connected to, respectively, said end portion of each of said plurality of elongated extension members.

2. The implantable device of claim 1, further comprising a plurality of sleeves, each removably covering at least a portion of said plurality of elongated extension members, respectively.

3. The implantable device of claim 2, further comprising a plurality of tips, each removably covering at least a portion of said plurality of sleeves and at least an end portion of said plurality of elongated extension members, respectively.

4. An implantable device, comprising:
a body member having a top portion, a bottom portion, and a longitudinal axis extending through said top portion and said bottom portion;
at least first and second elongated extension members on opposite sides of said body member, each (i) having an end portion, (ii) extending from said top portion, and (iii) having a longitudinal axis extending therethrough;
at least third and fourth elongated extension members, each (i) having an end portion, (ii) extending from said bottom portion, and (iii) having a longitudinal axis extending therethrough;
at least one suture, connected to at least one of said elongated extension members, respectively, at said end portion thereof;
at least one sleeve, covering a portion of at least one of said elongated extension members with said suture extending outwardly therefrom, respectively; and
at least one tip, covering and connected to said at least one sleeve and said elongated extension members, respectively, at said end portion thereof, with said suture extending outwardly therefrom, respectively.

5. The implantable device of claim 4, wherein said body member has a generally trapezoidal shape.

6. The implantable device of claim 4, wherein said longitudinal axis of said at least first elongated extension member is approximately colinear with said longitudinal axis of said at least second elongated extension member.

7. The implantable device of claim 4, wherein said longitudinal axis of said at least third elongated extension member is approximately parallel to said longitudinal axis of said at least fourth elongated extension member.

8. The implantable device of claim 4, wherein at least one sleeve is color-coded.

9. The implantable device of claim 4, wherein at least one suture forms a loop.

10. A surgical tool, comprising:
a shaft having a proximal end, an intermediate hook-shaped portion, and a distal end opposite said proximal end; and
at least one T-shaped slotted portion in said distal end of said shaft.

11. The surgical tool of claim 10, further comprising at least one L-shaped slotted portion in said distal end of said shaft.
